(19) United States
(12) Patent Application Publication
(10) Pub. No.: US 2024/0354647 A1
(43) Pub. Date: Oct. 24, 2024

(54) SYNTHETIC TABULAR NEURAL GENERATOR

(71) Applicant: The Cleveland Clinic Foundation, Cleveland, OH (US)

(72) Inventors: Hooman H. Rashidi, Pepper Pike, OH (US); Brian Rubin, Orange Village, OH (US); Bo Hu, Solon, OH (US); Samer Albahra, Cleveland, OH (US)

(21) Appl. No.: 18/640,197

(22) Filed: Apr. 19, 2024

Related U.S. Application Data
(60) Provisional application No. 63/460,642, filed on Apr. 20, 2023.

(51) Int. Cl. G06N 20/00

(52) U.S. Cl. CPC G06N 20/00

(57) ABSTRACT

A tabular synthetic data generation method comprising generating a plurality of synthetic datasets based on an empirically collected dataset according to a plurality of different models. The plurality of synthetic datasets are scored based on the respective automated machine learning (Auto-ML) analysis of a trained machine learning system based on the generated synthetic data. Identifying an optimal one of the generated synthetic datasets based on the Auto-ML scores. The Auto-ML scores can be based on analysis of machine learning systems trained on the empirically collected dataset, the generated synthetic datasets, and a tertiary ML analysis.

### CROSS-REFERENCE TO RELATED APPLICATIONS

This application claims priority to U.S. Provisional Patent Application No. 63/460,642 filed Apr. 20, 2023 and entitled “SYNTHETIC TABULAR NEURAL GENERATOR”, the entirety of which is incorporated herein by reference.

### BACKGROUND

Recently, the quantity of data generated in the healthcare sector has exponentially increased. The use of electronic health records (EHR) along with digital medical and pathology data are the major source of this medical data expansion, which provides a wealth of information and possibilities for advancing research and innovation. This helps improve patient care and provides more cost-effective delivery platforms within this domain.

However, healthcare data is typically governed by stringent regulations involving the Health Insurance Portability and Accountability Act (HIPPA) in the United States, and the General Data Protection Regulation (GDPR) in Europe, which prevents it from being readily accessible to the broader research community. Even when such data is available, investigators often need to submit appropriate proposals to regulatory committees (e.g., Institutional Review Board) to ensure protection of such data and their associated patient privacy before conducting research. In many cases, this is a lengthy process and severely delays the pace of research, especially for pilot studies. These delays help explain the sluggish adoption of new tools within medicine, particularly those related to machine learning because of their dependency on training systems with large amounts of data.

Synthetic data can help solve these data access challenges. Synthetic data is “new data” generated from a real data counterpart (e.g., data having been empirically collected). Compared to de-identified real data, which can be theoretically re-identified, synthetic data is not associated with a real patient or subject. The synthetic data is generated based on shared mathematical relationships that enable a new acquired function (i.e., a model) to generate the new data. The synthetic data can closely resemble its real data counterpart by retaining the statistical characteristics and/or patterns of the real data. Further, the synthetic data can eliminate patient privacy concerns because it does not represent any real individual patient. Therefore, the use of synthetic data has little to no privacy risks. Additionally, synthetic data can expand sample sizes several-fold, which can significantly reduce the data collection cost and also provide great statistical power (e.g., especially for rare disease or other limited data domains).

In short, synthetic data generation can create original new datasets to be used in analysis, innovation, advance research, and quality assurance studies without jeopardizing patient privacy or flouting legal requirements. Synthetic data lowers entry barriers for healthcare research and innovation by enabling researchers to test their pilot studies, train their algorithms, help simulate various clinical scenarios/situations in the absence of real patient data, and the like. Such an approach can expedite the start of most pilot projects and increase the number of new ideas and clinical studies, while minimizing current temporal and bureaucratic barriers.

While there are different methods, techniques, and platforms to generate synthetic data, many of these require machine learning and statistical expertise, along with coding knowledge and software engineering knowhow. Further, regardless of the platform employed, no single approach can adequately address all our tabular data needs and their intrinsic variabilities and limitations.

### BRIEF SUMMARY

According to one example of the present disclosure, a method comprises: obtaining an empirically collected dataset; generating a synthetic dataset based on the empirically collected dataset; performing an automated machine learning (Auto-ML) analysis of the synthetic dataset; and generating an Auto-ML score of the synthetic dataset based on a result of the Auto-ML analysis.

In various embodiments of the above example, the method comprises generating a plurality of synthetic datasets according to a plurality of different synthetic data generation methods, wherein the Auto-ML analysis is performed, and the Auto-ML score is generated, for each of the plurality of synthetic datasets; the plurality of different synthetic data generation methods comprise single function and multi-function models; the plurality of synthetic data generation methods comprise single function and multi-function versions of each of Gaussian copula, copula-GAN, CT-GAN, and TVAE models; the method further comprises identifying an optimal one of the plurality of synthetic datasets based on the Auto-ML scores; the method further comprises validating the plurality of synthetic datasets based on the Auto-ML scores; performing the Auto-ML analysis of the synthetic dataset comprises: training a synthetic data machine learning system with a training portion of the synthetic dataset, training a real data machine learning system with a training portion of the empirically collected dataset, inputting a testing portion of the synthetic dataset to the trained synthetic data machine learning system, inputting a testing portion of the empirically collected dataset to the trained real data machine learning system, and inputting the testing portion of the empirically collected dataset to the trained synthetic data machine learning system, wherein the Auto-ML score is based on a comparison of the outputs of the trained real data and synthetic data machine learning systems; the Auto-ML score is determined as 1−(|AUCsr−AUCrr|+|AUCss−AUCsr|), where: AUCsr is a value representing an area under a receiver operating characteristic curve (AUC) of an output of the trained synthetic data machine learning system from inputting the testing portion of the empirically collected dataset, AUCrr is a value representing an AUC of an output of the trained real data machine learning system from inputting the testing portion of the empirically collected dataset, and AUCss is a value representing an AUC of an output of the trained synthetic data machine learning system from inputting the testing portion of the synthetic dataset; the method further comprises generating a pre-ML score based on a statistical comparison of the empirically collected dataset and the synthetic dataset; the method further comprises generating a final score by averaging the Auto-ML score and the pre-ML score; and/or generating the synthetic dataset comprises: splitting the empirically collected dataset into a plurality of classification groups, and applying a synthetic data generation method to each of the plurality of classification groups.

### BRIEF DESCRIPTION OF THE SEVERAL VIEWS OF THE DRAWING

FIG. 1 illustrates an example synthetic tabular neural generator method of the present disclosure.

FIG. 2 illustrates an example control arm method of the present disclosure.

FIG. 3 illustrates an example synthetic data arm method of the present disclosure.

FIG. 4 illustrates an example synthetic tabular neural generator machine learning (STNG ML) scoring method of the present disclosure.

FIG. 5 illustrates an example computing system of the present disclosure.

FIGS. 6A-61 illustrate example STNG ML scores of different machine learning models of the present disclosure.

FIG. 7 illustrates comparisons of the real and STNG datasets of the present disclosure.

### DETAILED DESCRIPTION OF THE DRAWING

Considering the above, the present disclosure relates to a synthetic tabular neural generator (STNG) that utilizes machine learning (ML) to identify the optimal performance among a plurality of single and multi-function neural and non-neural network synthetic data generators.

### Synthetic Data Generation

Synthetic data generation techniques can be generally divided into probability distribution methods and neural network methods. Synthetic data generation can be accomplished using a number of different methods and techniques. For example, some generation methods start by estimating a probability distribution of the real data, and then draw random samples from the distribution as the synthetic data. Another method for estimating the probability distribution is the Gaussian copula-based method, where a joint distribution of the variables in the dataset is estimated with a Gaussian Copula model, reflecting the dependency (inter-correlation) between each variable in the data set. A chained-equation approach estimates the conditional distribution of each variable given other variables, and generates the synthetic values of the variables sequentially.

Some methods may involve neural network-based approaches such as Generative Adversarial Network (GAN) and variational autoencoders (VAE). Generative Adversarial Network (GAN) based approaches jointly train two neural networks-one to generate the synthetic data and another to discriminate the real data and the synthetic data generated by the first network. These two neural networks are adversarial and compete against each other to achieve optimal performance. The GAN-based approaches can be particularly beneficial in generating synthetic tabular data, electronic health records (EHR), texts, and images. Additionally, variational autoencoder (VAE) methods include the tabular variational autoencoder (TVAE) and oblivious variational autoencoder (OVAE), which can sometimes outperform the GAN-based approaches in certain empirical studies.

Synthetic data generation can also be achieved using various open source and commercial platforms. For example, the Synthetic Data Vault (SDV), created by MIT's Data to AI Lab, is the largest open-source ecosystem for synthetic data generation and evaluation. SDV implements various non-deep learning copula and certain deep learning-based models for synthetic data generation, and also provides an evaluation framework to assess the quality of such synthetic data. Other open-source platforms include the R packages synthpop and SimPop and the Python package DataSynthesizer. Commercial platforms available for synthetic data generation include MDClone, Syntegra, and Octopize MD.

STNG variations on each of the above techniques further involves splitting the real data into a plurality of classification groups, and generating synthetic data for each classification group according to the generation method. This effectively generates subsets of synthetic data according to the generation method for each real data set, in contrast to applying the entire real data set to the generation method.

Using the methods and systems described herein, generation of synthetic tabular data is more accessible to researchers and clinicians that seek data for pilot studies or any other studies. Particularly, the methods and systems described herein expedite the generation of synthetic data while still preserving original characteristics of real data. Additionally, small datasets of real data could be expanded, which can significantly reduce the data collection cost and also provide great statistical power. Still further, the best method for generating synthetic data can be quickly and accurately identified.

### Synthetic Data Generator and Validation

Briefly, the present system and method generate and validate synthetic data based on a non-biased (i.e., “no assumption”) approach to tabular synthetic data generation. It incorporates and concurrently auto-validates a plurality synthetic data generators. More particularly, the present system and method can use real patient data to train a machine learning system. The trained machine learning system can be used as a ground truth for the testing of machine learning systems trained on synthetic data. Using the single and multi-function neural and non-neural network synthetic data generators described above, the present system and method then generates a multitude of synthetic datasets from the real patient data. The synthetic datasets can be split into training data and testing data. The training data can be used to train a synthetic data machine learning system. The synthetic testing data and the real testing data can be input into a trained synthetic data machine learning system to validate and analysis the output. The analysis of the outputs of the multitude of synthetic datasets are compared to identify the optimal performance among the plurality of single and multi-function neural and non-neural network synthetic data generators. The analysis of the synthetic datasets include scoring the synthetic datasets compared to the real data set, allowing a user to determine which synthetic dataset is the best representation of the real data set.

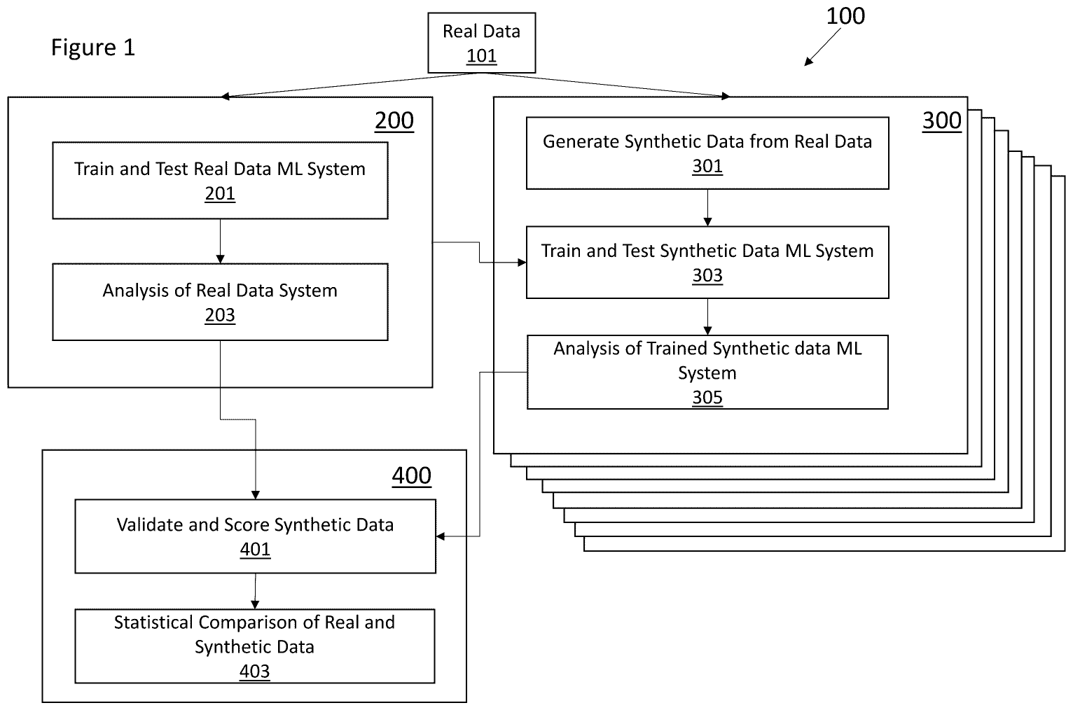

With reference to FIGS. 1-3, the generation and validation of synthetic data will be described in more detail. With particular reference to FIG. 1, the method first obtains an empirically collected real data set 101. The real data 101 set can be collected from a single or plurality of institutions, studies, or the like. The real data 101 is used both in a control arm 200 of the method to train a machine learning system according to a ground truth, and in a synthetic data arm 300 to generate and validate synthetic data sets.

With respect to the control arm 200, the real data 101 is used to train and test 201 a real data ML system. This is further described below, but briefly includes splitting the real data set into real training data 204 and real testing data 206. The real training data 204 may have a balanced target class (e.g., equal number of observations from each output class) and the sample size per class is half of the size of the smallest class in the original real dataset. The observations per class may be selected randomly. In some embodiments, the total sample size of the real training data 204 may be capped, for example to 500, in order to train all ML systems in a manageable time period. The remaining observations are included in a secondary generalization test set.

After the real data ML system 210 is trained using the real training data 204, the real data ML system is analyzed 203. The analysis may include testing the real data ML system 210 with the real testing data 206 from the real data set 101. This analysis determines whether the trained ML system performs correctly and how well the system performs, for example, by identifying the number of true outputs relative to the ground truth.

With respect to the synthetic arm 300, the real data 101 is used to generate synthetic data 301. This generated synthetic data 301 is used to train and test 303 a synthetic data ML system 310. That is, as described in more detail below, the synthetic data 301 is split into a synthetic training data 304 and synthetic testing data 306. A synthetic data ML system 310 is then trained with the synthetic training data 304 and tested with the synthetic testing data 306. An analysis of the trained synthetic data 305 evaluates whether the synthetic data ML system 310 was trained correctly and how well it performs. The analysis can include inputting the synthetic testing data 306 split from the generated synthetic data 301, and scoring the accuracy of the synthetic data ML system 310 output relative to the ground truth.

In addition to the synthetical testing data 306, the synthetic data ML system 310 may also be tested with the real testing data 206 to determine the model's true generalizability on the real data 101. That is, system may perform an Auto-ML technique to confirm accuracy of the synthetic data ML system on both the real data 101 and the synthetic data 301. With this approach, the better the performance of the synthetic data ML system 310 with real data 101 (particularly as compared with the real data ML system), the more accurate the generation method of that synthetic data 301. Put differently, the synthetic data 301 may be considered an accurate representation of the real data 101 if the synthetic data ML system 310 treats the synthetic data 301 as well as real data 101 and the real data ML system 210; thus, the generation method for that synthetic data 301 may be considered an appropriate method for generating synthetic data based on that particular real data 101.

More particularly and with reference to the analysis arm 400, the true performance of each synthetic data 301 is evaluated by comparing its synthetic data ML system 310 prediction performances 305 with the performance of the real data ML system 210. This evaluation is used to validate and score 401 the synthetic data and generation method thereof.

In some embodiments, the assessment of the trained machine learning systems can include determining a Matthews correlation coefficient (MCC), receiver operating characteristic (ROC), an area under the ROC curve (AUC), and the like for identifying the success rate of each trained ML system's ability to correctly classify the testing data 206, 306 described above. A score and/or ranking for each synthetically generated dataset may then be generated based on the assessment. The score and/or ranking may additionally or alternatively be based on a statistical comparison 403 between the real 101 and synthetic data 301. A report of the scores and/or assessments of each synthetic data 301 may then be output to a user. Based on these scores and/or assessments, the user may select the synthetic data 301 from any of the generation methods (e.g., the synthetic data 301 having the highest score) for further use, such as for additional machine learning studies.

As suggested above, the method of the synthetic data arm 300 is repeated for each of a plurality of synthetic data 301 generated according to a plurality of generation methods. For example, synthetic data 301 may be generated for each of four open-source single function models and four multi-function models, producing eight sets of synthetic data 301. Depending on the embodiment, the size of each synthetic data 301 (e.g., the number of data entries) generated is the same as the real data 101 (i.e. a 1:1 ratio). However, any ratio may be utilized. For example, the synthetic data 301 may be 2, 3, 4 5, or more times the size of the real data 101. The ability to expand size of the synthetic dataset can be particularly beneficial if the real data 101 is of a limited sample size. Further, increasing the sample size may improve the reliability of the performance measures when applied for machine learning. Ultimately, this synthetic data generation gives rise to eight competing synthetic tabular data generators 301. For each real data 101, no preliminary identification of the best generator is made. That is, the synthetic data generator of the present disclosure makes no assumptions to which generation method is best, and performs multiple generations regardless.

In one embodiment, eight synthetic data generators produce corresponding synthetic data based on Gaussian copula, copula-GAN, CT-GAN and TVAE synthetic data generation methods, respectively. Four of these are the open-source generic single function methods while the other four are modifications of these methods through a multi-function approach (herein known as the STNG version of each). However, other embodiments may use more, less, and/or different synthetic data generators.

Each of the plurality of sets of synthetic data 301 are then tested and scored so that one or more optimal synthetic datasets can be selected and used. This validation process can be based on common statistical features which compares the synthetic datasets to the real dataset. However, within the STNG platform in addition to the aforementioned, a more rigorous validation can also be performed which is based on its embedded fully automated machine learning (Auto-ML) analysis platform.

### Control Arm

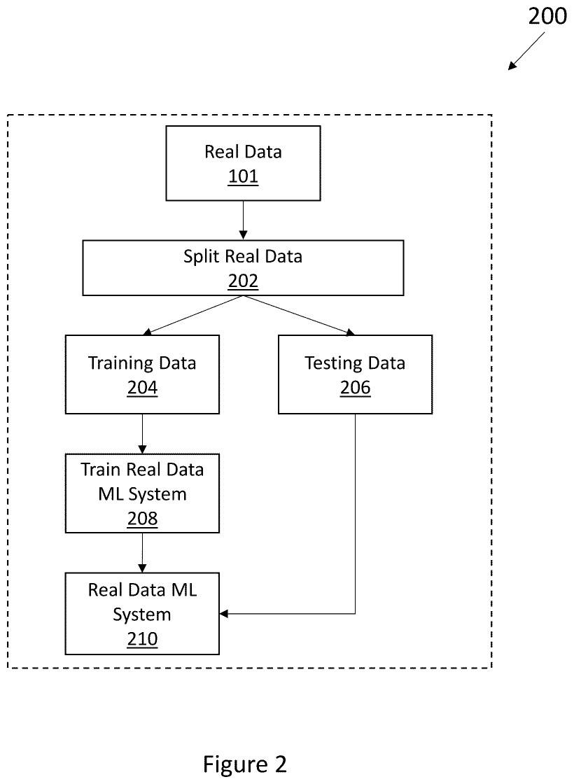

With further regard to the control 200 and FIG. 2, the control arm method includes splitting 202 the real data 101 into real training data 204 and real testing data 206. The real data 101 can comprise tabular data, i.e., data that can easily be display in columns or tables. In a healthcare sense, this data may include patient and disease data such as age, weight, height, race, sex, body mass index, physiological data, whether particularly symptoms are present, date when symptoms started, and the like.

The real data 101 can be split 202 using the Pareto ratio of 80:20 or any other ratio, e.g., 70:30. The splitting 202 of real data can be randomized or comprise any other splitting method. In one particular example, a real data 101 comprises one thousand entries in which three-hundred cases are associated with patients diagnosed with a disease and seven-hundred entries are associated with patients without the disease. The real data 101 is split into real training data 204 comprising two-hundred entries with the disease and two-hundred entries without the disease; and real testing data 206 comprising one-hundred entries with the disease and five-hundred entries without the disease.

As discussed above, the real training data 204 is used to train 208 a real data ML system to develop a real data machine learning system 210. Considering the above example, the real data ML system 210 can be trained to identify whether or not a patient has the disease. The real data ML system 210 can be trained using supervised, unsupervised, and/or semi-supervised learning. This real data ML system 210 is used as a ground truth for the synthetic data trained machine learning systems 310, described below. That is, the real testing data 206 is used to test and analyze the real data trained machine learning system 210 and produce a baseline or ground truth.

Using the real testing data 206 to analyze the real data ML system 210 allows the user to determine if the real data ML system 210 is performing correctly. For example, the real test data 206 is input into the real data ML system 210, and the output is identified as correct or incorrect. For the example described above, real testing data 206 can be used to determine if the real data ML system 210 properly identifies whether an inputted patient entry from the real testing data 204 has a disease. The real data ML system 210 is given based on this analysis, which is used for comparison with ML systems 310 trained by synthetic data. As discussed herein, this comparison can be used to determine if a machine learning system trained with synthetic data 310 performs similarly to the real data trained system 210, thus suggesting a valid synthetic data set. The real testing data 206 is also used to test a synthetic data ML system trained with synthetic data 310, further suggesting a validity of that synthetic data 301.

### Synthetic Data Arm

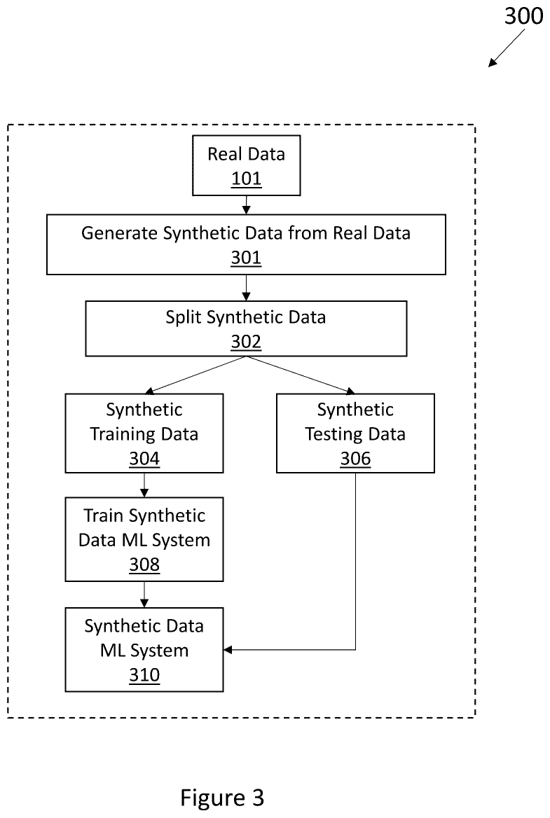

With further regard to FIG. 3, the method of the synthetic data arm 300 uses the real data 101 to generate synthetic data 301. As described above, synthetic data 301 can be generated using any method, including those that are open-source, proprietary, single, and/or multi-function models. As discussed above with respect to real data 101, the synthetic data 301 can be split 302 into synthetic training data 304 and synthetic testing data 306 according to any relationship and at any size ratio with respect to the real data 101. The synthetic data 301 can be split 302 at a different ratio than the real data 101.

Similar to the control arm 200, the synthetic training data 304 is used to train 308 a synthetic data ML system 310 according to any training method and the synthetic testing data 306 can be used to test the synthetic data ML system 310. The outputs of the synthetic data ML system 310 can then be analyzed 305 to score the synthetic data ML system 310 based on its accuracy.

Additionally, the synthetic data ML system 310 is further tested with the real testing data 206 (or the entirety of the real data 101) according to the Auto-ML technique by applying the real data 101, 206 to the synthetic data ML system 310 and determining the accuracy of the output. Comparison of the accuracy of the outputs of the synthetic data ML system 310 based on the real testing data 206 and synthetic testing data 306 with each other, and to the output of the real data ML system 210 based on the real testing data 206, indicates the validity of the synthetic data 302. The synthetic data ML system 310 can further be scored based on analysis 305 these test comparisons.

### Auto-ML Analysis and Validation Scoring

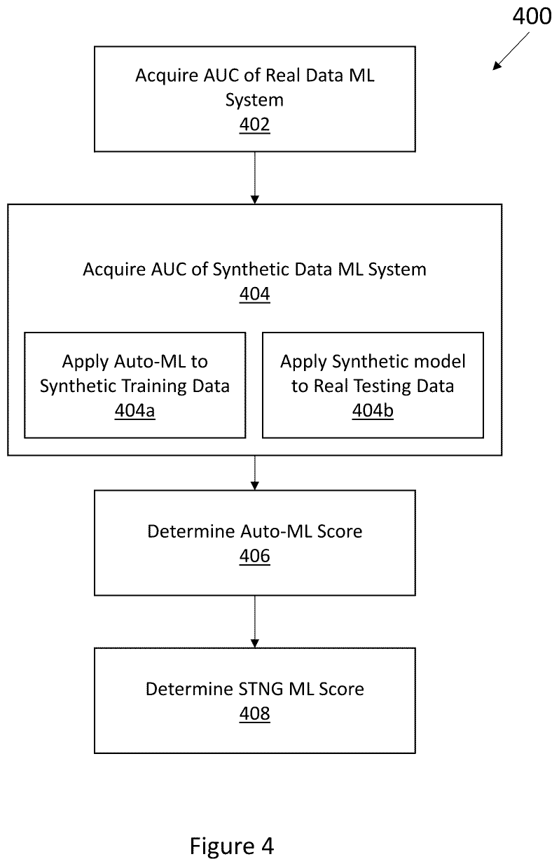

With reference to FIG. 4, analysis 203 of the real data ML system 210, analysis 305 of the synthetic data ML system 310, and scoring 401 and comparison 403 of the synthetic data 301 and real data 101 is described in more detail below.

Particularly, any of the aforementioned ML systems 210, 310 may be binary or multi-class classification tasks, for example, including but not limited logistic regression, naïve Bayes, k-nearest neighbor (KNN), support vector machine (SVM), and multi-layer perceptron (MLP) neural network. According to one embodiment, for each classification task, validation incorporates the aforementioned supervised ML systems, two scaling methods, certain options in feature selections and various hyperparameter optimization search methods (as shown in Table 1). For feature selection, the top 30%, 50%, 80% or 100% of the features based on their F test statistics may be applied. The features could be on the original scale or be scaled to have means of zero and standard deviations of one (i.e., standardization scaling). The hyperparameters for each classification can be searched randomly or through a grid method except for naïve Bayes (since it precludes any hyperparameters to tune).

Based on the combinations of the classification supervised algorithms noted above and their options (i.e., scaling, feature selection and hyperparameter tuners), a total of 80 validations can be deployed for each of the synthetic datasets generated, which equates to 74,480 total number of ML models generated and evaluated for each. Therefore, the total number of ML models generated in all 8 synthetic data generators for the entire full run is equal to 640 validations, which translates to 595,840 total number of ML models generated and evaluated, allowing for a comprehensive and unbiased model selection for any given dataset.

TABLE 1
Classification algorithms and their options
implemented in STNG's Auto-ML module

| | Options |
|---|---|
| Supervised | Logistic regression, naïve Bayes, |
| Algorithms | k-NN, SVM & MLP |
| | neural network |
| Features to be used | 30% vs. 50% vs. 80% vs. 100% |
| Scaling | Standardization Scaling versus no scaling |
| Hyper-parameter search | Grid search and random search¹ |

¹Not applicable for naïve Bayes classification

The evaluation procedures described above are applied to each generated synthetic data 302 (e.g., 8 sets corresponding to 8 generation methods), which allows objective comparison of the true performance of each synthetically generated data 302 to each other and their real data-based ML system performance. A STNG ML score is determined to objectively assess the performance of each of these synthetic ML systems 310 and to help highlight the best synthetic generator. With respect to FIG. 4, according to one embodiment the STNG ML score is determined as follows:
1. Acquire 402 highest area under the receiver operating characteristics curve (AUC) from applying real testing data 206 to the real data ML system 210 (AUCrr);
2. Acquire 404 highest AUC of the synthetic data ML system 310 by:
   a. Acquiring highest AUC from applying synthetic testing data 306 (AUCss); and then
   b. Acquiring highest AUC from applying real testing data 206 (AUCsr);
3. Determine “Auto-ML score” 406 as 1−(|AUCsr−AUCrr|+|AUCss−AUCsr|);
4. Determine “STNG ML score” 408 by averaging the “Auto-ML score” and a pre-ML similarity score. The pre-ML similarity score may be determined by statistical analysis of the synthetic data 301 relative to the real data 101, without comparison to other generation methods.

The STNG ML score objectively incorporates the post ML AUC measures and the pre-ML statistical similarity measures which addresses any limitations that each score may have had when assessed in silo. Since AUCsr and AUCrr are derived by applying the real data ML system 210 and synthetic data ML system 310 to the real testing data 206 and the synthetic testing data 306, respectively, the term |AUCsr−AUCrr| can be considered a measure of the difference in the ML system trained from the real training data 204 and synthetic training data 304. Meanwhile AUCss is the AUC of the synthetically trained ML model, being applied to the real data testing data 206 and the synthetic testing data 306. Thus |AUCss−AUCsr| can be considered as a discrepancy caused by the difference in the real testing data 206 and synthetic testing data 306.

Considering the above, the Auto-ML score represents a combination of model difference and dataset difference, which can be interpreted as how well a synthetic data 302 is able to replicate the ML prediction performance that will be derived from the real data 101. Based on the Auto-ML score, the final STNG ML score can yield a value between 0 and 1, with a value of 1 indicating a perfect replication and 0 signifying no similarity. In some embodiments, the AUCs and the ML metric may not be computed for synthetic data 302 with a class size of less than 80 observations.

Of course, other metrics and relationships between metrics may be utilized as a representative score of each synthetic dataset, generated in the statistical comparison of real and synthetic data step 403. For example, in addition to ROC-AUC, other common performance metrics for classification can include accuracy, F1 score, and Matthew's correlation coefficient (ϕ). Users can choose their own preferred metrics to rank the synthetically generated datasets.

The extension of the STNG ML score for multi-class classification can be performed as follows. For the ROC-AUC, the one-vs-rest is used along with the macro-averaged metrics for the confusion matrix-based performance measures.

In addition to the ML evaluation metric described above, various standard non-ML statistical metrics can be used to evaluate the quality and utility of the synthetic datasets as compared to their real data counterparts. These can be grouped into three categories: univariate, bivariate, and overall metrics.

The univariate metrics can apply to the real and synthetic values of each variable to assess whether their marginal distributions are similar, which include both graphical and numerical evaluations. Regarding graphical evaluations, the histograms, and the cumulative summations of each variable in the real and synthetic datasets can be compared. The numerical evaluations can include standard descriptive statistics such as means, medians, ranges, and standard deviations for each variable. Furthermore, the Kullback-Leibler (KL) divergence can be determined as a measure of the similarity between the real and synthetic values in terms of their probability mass functions. Further, the Kolmogorov-Smirnov (KS) test statistic can be derived by comparing the two cumulative density functions. The KL divergence and the KS statistic can then be averaged across all variables into composite scores to reflect the overall similarity across all variables.

The bivariate metric can involve the pairwise correlation difference (PCD), which is determined as |Corrᵣ−Corrₛ|_{F}, where Corrᵣ and Corrₛ are the matrices of pairwise correlations of real and synthetic datasets, respectively, and ∥_{F} represents the Frobenius form. Thus, PCD measures the closeness of the overall correlation structures of the real and synthetic datasets. Smaller values of PCD imply closer correlation structures between the two datasets. Heatmaps can also be provided to show the correlation matrices of the real and synthetic datasets, respectively for each PCD.

The overall metrics for each of the synthetic data 302 can also be independently evaluated through statistical performance metrics including the following:
a. Likelihood metrics comparing the two datasets by fitting the real data to a probabilistic model (such as Bayesian network or Gaussian mixture models), and then determining the likelihood that the synthetic datasets follow the fitted model;
b. Distinguishability metrics characterize the ability to distinguish the real and synthetic data records. In one example, the real dataset and the synthetic dataset are stacked together with a binary indicator assigning whether each data record is from the real dataset or the synthetic dataset. A binary classifier can be trained to the stacked dataset with the binary indictor as the output. The classification performance in discriminating between the real and synthetic observations can be used as a distinguishability metric. A logistic regression model can be applied as the classifier with classification accuracy as the metric. A lower accuracy implies that the real and synthetic observations are more difficult to separate, and thus they are more similar. The propensity mean square error is another distinguishability metric. The estimated probabilities from the logistic regression model are referred to as propensity scores, and the mean squared propensity score error is determined according to

$$\frac{1}{N} \sum {( {p}_{i} - 0.5 )}^{2} ,$$

where pᵢ is the propensity score for the ith record in the stacked dataset;
c. Log-cluster metric measures the similarity of the underlying dependency structure in terms of clustering. Instead of applying a classifier to discriminate between the real and synthetic observations in the stacked dataset, clustering analysis is performed. A metric is determined to reflect the distribution of the synthetic dataset across the different clusters. If the allocation to the different clusters is similar for synthetic and real, then this suggests similar distributions;
d. Cross-classification metric measures the similarity of the prediction performances from the synthetic and real datasets. In one example, the real data 101 is first split into real training data 204 and real testing data 206. A classifier is then trained on the real training data 204 and applied to the real testing data 206 and a synthetic data 301, where the ratio of the classification performances (e.g., AUC) is the (real-synthetic) cross classification metric. The cross-classification can be derived the other way around, with a classifier trained from the synthetic training data 304 and the performance ratio calculated from the synthetic testing data 306 and the entire real data 101, which can be called as the (synthetic-real) cross classification metric. In general, the closer the cross-classification metrics are to 1, the better the synthetic data are.

The cross-classification metric differs from the STNG ML metric in two aspects. First, a classifier is pre-specified in determining the cross-classification metrics while the derivation of the STNG ML metric employs an auto-ML pipeline. Secondly, the determination of the cross-classification metrics comparing the performance ratio of the synthetic testing data 306 and real data 101 by applying a same classifier to them, which instead captures the prediction differences due to the use of different datasets. On the other hand, the calculation of the STNG ML metric involves splitting both the synthetic data 301 and real data 101 into their training and test sets, respectively. Since different classifiers may be derived from the synthetic data 301 and real data 101, the STNG ML metric also captures the possible difference of the ML systems, which is thus a more comprehensive metric and closer to real practice than the cross-classification metric.

TABLE 2
Summary of the statistical utility metrics
for evaluating synthetic datasets

| Category | Metric | Value Range |
|---|---|---|
| Univariate | Mean, median, range, | |
| metric | standard deviation | |
| | Kullback-Leibler | Range between 0 and 1; lower |
| | divergence | values are better |
| | Kolmogorov-Smirnov | Range between 0 and 1; lower |
| | test statistic | values are better |
| Bivariate | Pairwise correlation | |
| metric | difference (PCD) | |
| Overall | Gaussian mixed | |
| metric | model likelihood | |
| | Propensity mean square | Range between 0 and 1; lower |
| | error (pMSE) | values are better |
| | Logistic distinguish | Range between 0 and 1; lower |
| | metric | values are better |
| | Cluster metric | |
| | Cross-classification | Values closer to 1 are better |
| | metric | |

### Implementation

The above method may be implemented as an end-to-end user system. In other words, a user may input real tabular data into the system and then receive a report of and access to a plurality of synthetically generated datasets based on the input real data. No prior machine learning, programming, or like expertise is required of the user to obtain and understand the validity of one or more synthetically generated datasets. Depending on the embodiment, such a system may be available locally (e.g., on-premises) or remotely (e.g., over the “cloud” 512, such as with a software as a service (SaaS) platform). Providing the system locally on-premises can help minimize online security concerns in accessing private data and can be installed and deployed through a simple Docker desktop application in one's private IT environment.

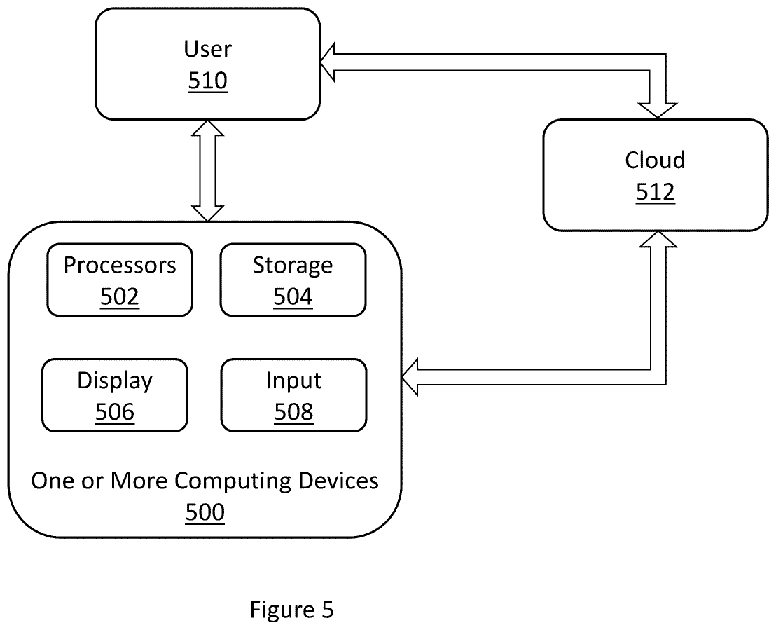

With respect to FIG. 5, one or more computing devices 500 may include one or more processors 502 and memories (storage, e.g., of a non-transitory type) 504 configured to implement the machine learning systems, generate the synthetic data, perform the analyses, output the analyses, store data, and the like described above. The computing devices 500 can also comprise displays 506 (e.g., monitors, tv, screen, a virtual/augmented headset, or the like), and inputs 508 (e.g., a mouse, keyboard, touchscreen, voice controls, virtual/augmented reality controllers, or the like). Further depending on the embodiment, the computing devices 500 can be mobile devices (such as a phone or tablet), a traditional computer or laptop, server, or the like. Further, any actions performed may be distributed among multiple computing devices 500 in any manner. When more than one computing device 500 is utilized, those computing devices may be connected in any manner (e.g., wired or wireless, via local networks, the Internet, and the like).

Any of the computing devices 500 may implement any portion of the method by a computer application including a user interface (UI) on the display 506. In this interface, the user can input real data 101 into the application, and the application can run the STNG method to generate the plurality of synthetic data and produce score/validation results. The analysis and scores of the generated synthetic data sets can be displayed, for example, in bar charts, indicating the ROC AUC, Auto-ML Score, and/or STNG ML Score for each of the synthetic testing data 306 and real testing data 206 for each generation method. The computer application can further rank the each synthetically generated data 302 based on these scores, and recommend the best scoring data (and corresponding generation method) to the user. In some embodiments, this ranking may be shown by the order of the displayed bar charts. The user may then further download, save, e-mail, or the like, the recommended synthetic data 302, or any of the other generated synthetic data they wish to use. The user may also generate additional synthetic data according to any of the desired generation methods within the application.

### Demonstrated Results

Table 3 lists a total of twelve datasets used in empirical studies conducted to validate the performance of the present disclosure. There are 9 datasets with binary output classes and 3 datasets for multi-class classification. The numbers of features (i.e., independent variables) within each of the datasets studied vary from 6 to 98 and their samples sizes vary from 280 to 13,611.

TABLE 3
Datasets used in the validation study

| | Number of | | | |
|---|---|---|---|---|
| | output | Number of | Sample | |
| Dataset | classes | features | size | Data source |
| Asthma | 2 | 98 | 2026 | National Health |
| attack | | | | and Nutrition |
| | | | | Examination Survey |
| Wisconsin | 2 | 9 | 697 | Kaggle |
| Breast | | | | |
| cancer | | | | |
| Breast | 2 | 20 | 280 | University of California, |
| cancer | | | | Irvine ML repository |
| recurrence | | | | |
| COVID19 | 2 | 88 | 362 | PRIDE repository |
| | | | | (project PXD021388) |
| Diabetes | 2 | 8 | 768 | Kaggle - National |
| | | | | Institute of Diabetes |
| | | | | and Digestive and |
| | | | | Kidney Diseases |
| Heart | 2 | 13 | 303 | Kaggle |
| disease | | | | |
| IBM | 2 | 50 | 1676 | Kaggle |
| employee | | | | |
| attrition | | | | |
| Oxide class | 2 | 8 | 383 | Dataset courtesy |
| | | | | of Professor Galen |
| | | | | Stucky at University of |
| | | | | California, Santa Barbara |
| Stroke | 2 | 10 | 4981 | Kaggle |
| Dry beans | 7 | 16 | 13611 | Kaggle |
| Diabetes | 3 | 17 | 2437 | National Health |
| | | | | and Nutrition |
| | | | | Examination Survey |
| | | | | (2015-2016) |
| Pancreatic | 3 | 6 | 590 | Kaggle |
| cancer | | | | |

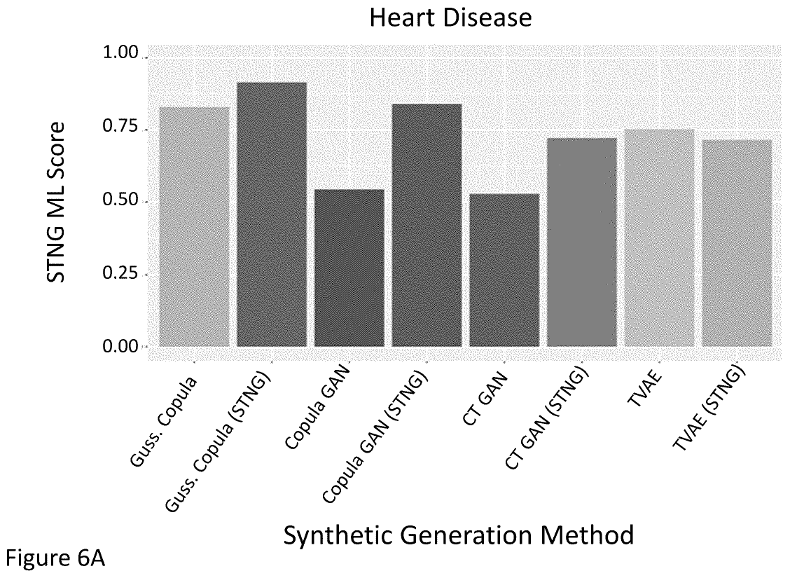

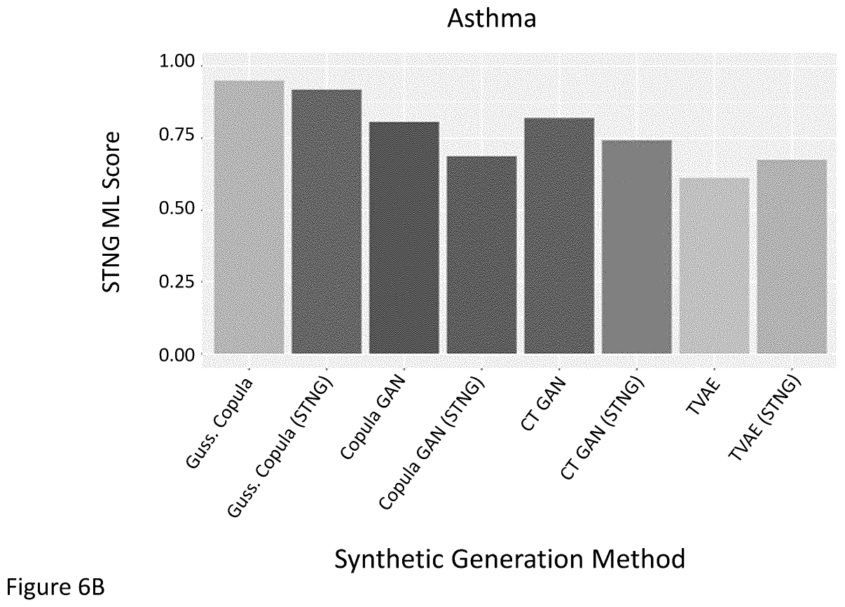

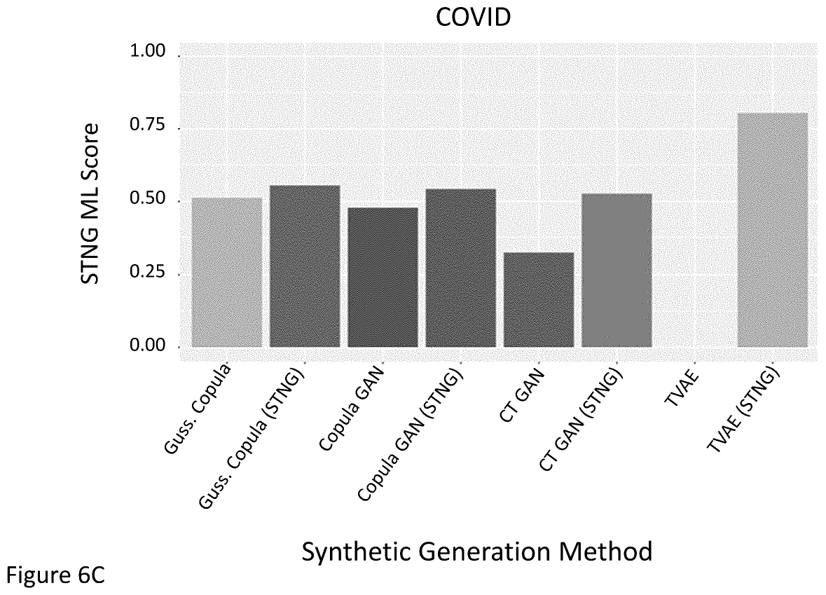

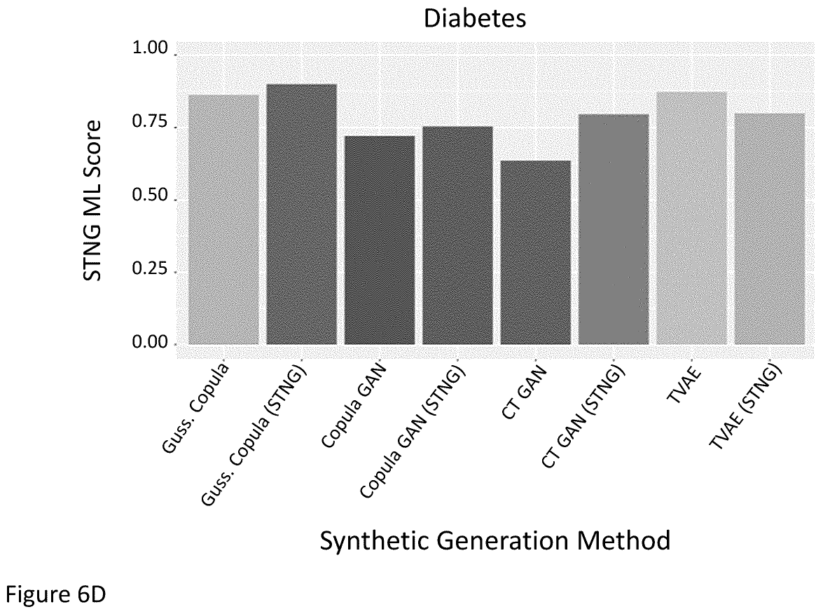

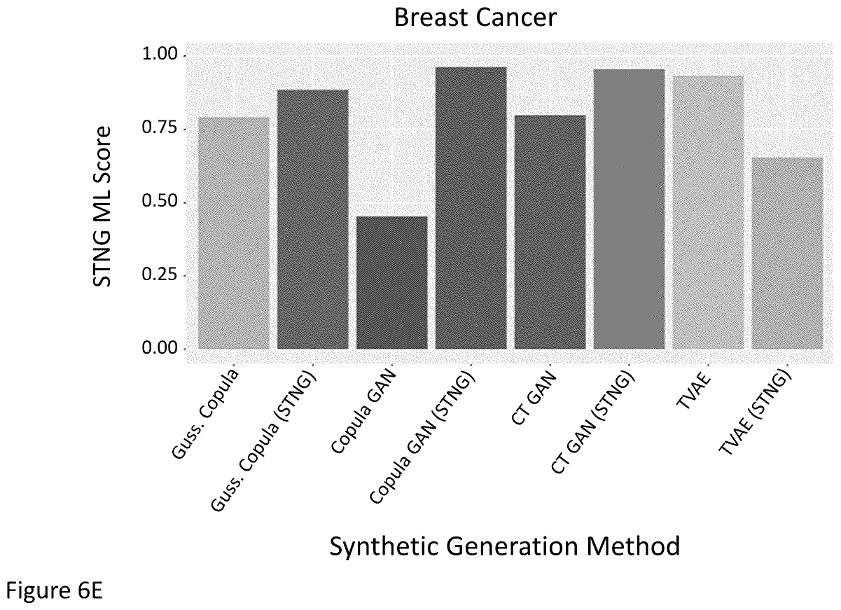

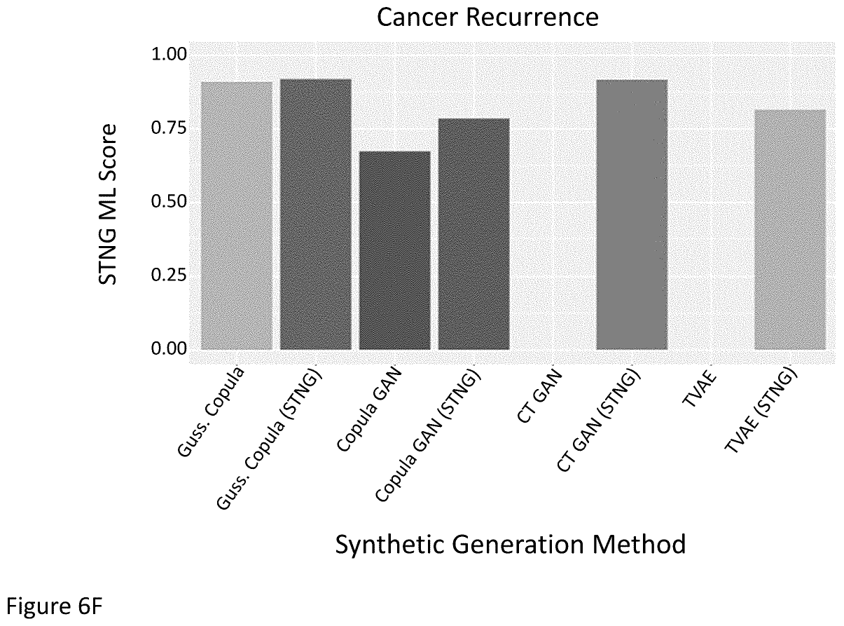

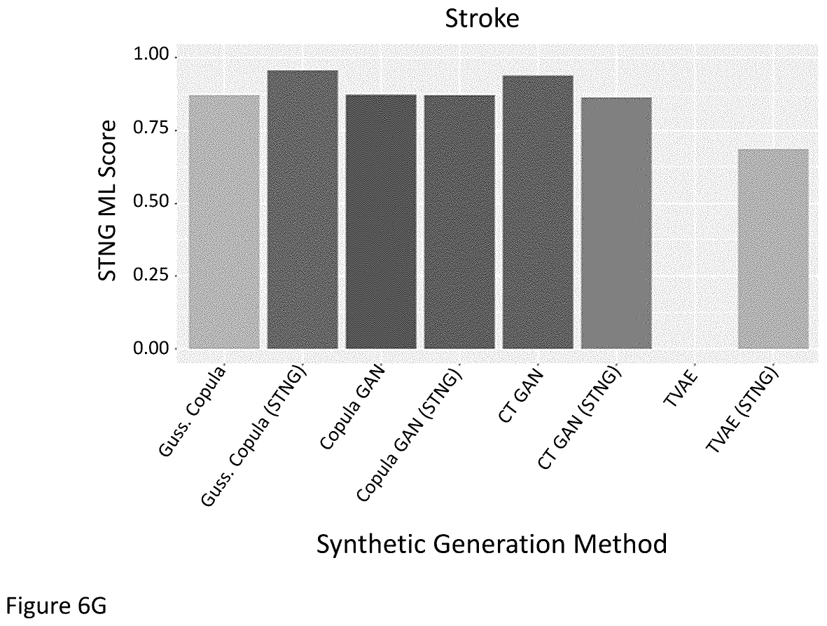

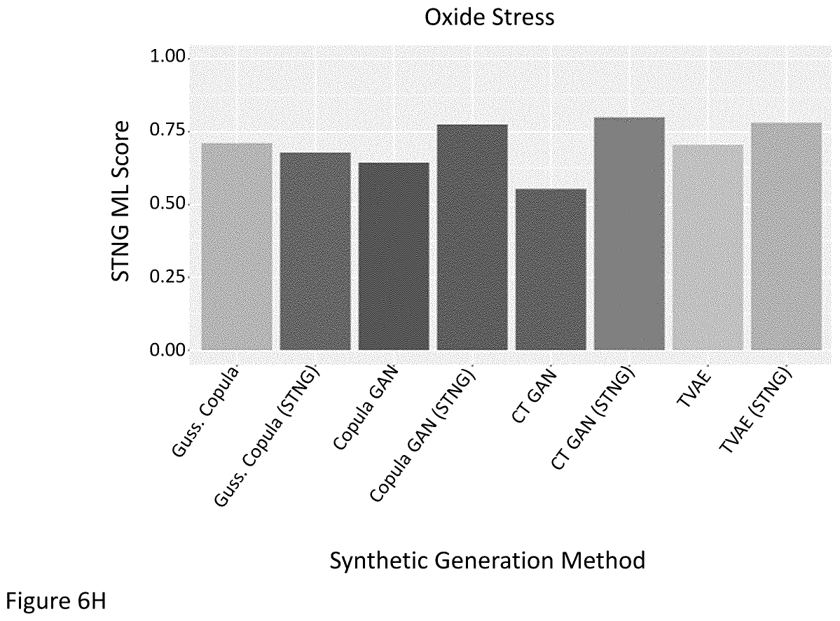

As shown in FIGS. 6A-6I (each corresponding to a different dataset among the 9 datasets for binary classification), STNG versions of synthetic data generators (splitting real data sets into a plurality of classifications to which the synthetic data generators were applied) outperformed the traditional synthetic data generators (in which the synthetic data generator is applied to the entire data set) on 8 of the 9 binary dataset studies. The synthetic datasets generated using the STNG Gaussian copula approach were found to be the top performers for five of the datasets based on the STNG ML performance scores. The STNG ML score was highest for each of the other three STNG neural network approaches, that is, STNG TVAE for the COVID19 dataset, STNG CT-GAN for the oxide dataset, and STNG copula GAN for the breast cancer dataset. For the asthma dataset, the generic Gaussian copula approach had the highest STNG ML score. Moreover, for four datasets, one or two of the generic approaches failed to generate synthetic datasets (as was mentioned earlier) since their synthetic data did not produce the minimum 80 observations per class that is required by the STNG Auto-ML process. In particular, the generic TVAE approach failed for the COVID dataset, the employee attrition dataset, the cancer recurrence dataset, and the stroke dataset. On the contrary, the modified STNG multi-function approaches did not have any failures.

Similar findings were observed from the results of the other three datasets with multi-class outputs. These observations demonstrate that overall, multi-function approach of STNG generally led to better performance outcomes than the generic approaches. Although the STNG multi-function synthetic data generators are generally the better performers, no assumptions are made about which generator is best for one's given data, which ultimately maximizes the likelihood of attaining the best performing synthetic dataset for a given study (regardless if it was generated through the STNG multi-function approach described herein or through one of the generic synthetic data generators). In other words, making no assumptions and using all synthetic data generators minimize bias, allowing the generation of better performing ML systems.

In addition to the STNG ML score noted above, the pre-ML statistical metrics also indicated that the synthetic dataset from the STNG Gaussian copula approach was generally the best synthetic dataset. As shown in Table, 4 its statistical metrics were all highest except for the SR cross classification, for which the STNG TVAE dataset had the highest value.

TABLE 4
Statistical evaluation metrics of synthetic heart disease datasets

| | Gaussian copula | | Copula GAN | | CT GAN | | TVAE | |
|---|---|---|---|---|---|---|---|---|
| Metric | Generic | STNG | Generic | STNG | Generic | STNG | Generic | STNG |
| KS statistic | 0.887 | 0.909 | 0.834 | 0.859 | 0.840 | 0.865 | 0.834 | 0.891 |
| Logistic | 0.736 | 0.897 | 0.360 | 0.411 | 0.234 | 0.487 | 0.224 | 0.415 |
| separation | | | | | | | | |
| GMM | 0.000 | 0.000 | 0.000 | 0.000 | 0.000 | 0.000 | 0.755 | 0.985 |
| likelihood | | | | | | | | |
| KL | 0.794 | 0.824 | 0.701 | 0.670 | 0.672 | 0.706 | 0.761 | 0.816 |
| divergence | | | | | | | | |
| PCD | 0.280 | 0.252 | 0.898 | 0.847 | 0.892 | 0.743 | 0.610 | 1.000 |
| pMSE | 0.907 | 0.960 | 0.651 | 0.689 | 0.513 | 0.773 | 0.480 | 0.689 |
| nLog cluster | 0.998 | 0.999 | 0.938 | 0.948 | 0.937 | 0.997 | 0.992 | 0.977 |
| Support | 0.926 | 0.928 | 0.888 | 0.898 | 0.878 | 0.909 | 0.740 | 0.789 |
| coverage | | | | | | | | |
| RS cross | 0.926 | 0.950 | 0.759 | 0.945 | 0.704 | 0.967 | 0.977 | 0.923 |
| classification | | | | | | | | |
| SR cross | 0.922 | 0.958 | 0.895 | 0.896 | 0.951 | 0.944 | 0.957 | 0.976 |
| classification | | | | | | | | |

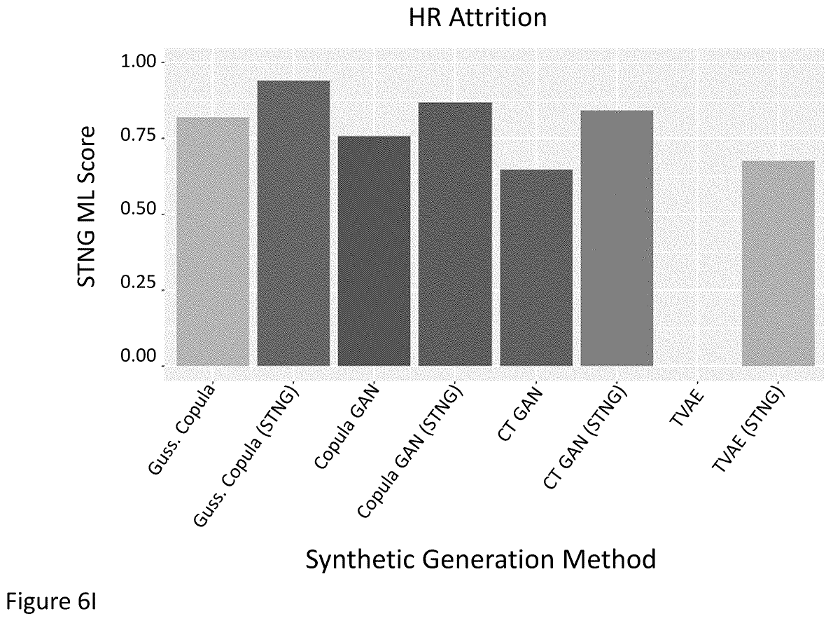

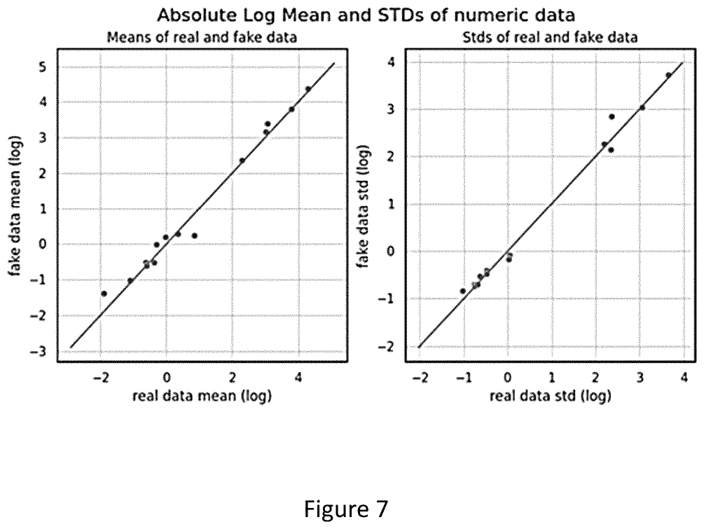

Once the synthetic dataset from STNG Gaussian copula was identified, further evaluations could be performed for comparing it with the real dataset based on common statistical metrics. For example, the absolute means and absolute standard deviations for real and synthetic heart disease datasets were calculated from the real dataset and were compared with the corresponding values from the synthetic dataset after log transformations. These relationships are illustrated in FIG. 7, which show a high degree of correlation. Furthermore, cumulative sum (cumsum) and quantile-quantile (QQ) plots were also derived for each variable in the real and synthetic dataset, showing generally consistent agreement except for a small deviation for the variable of trestbps. Regarding bivariate relationships, the pairwise correlations were shown for the same set of variables in the real and synthetic datasets. The difference of the pairwise correlations showed that the differences of the bivariate correlations were generally smaller than 0.1, and the correlations between the variable of trestbps and other variables were slightly higher in the synthetic dataset.

In an additional example, synthetic data was generated from empirically collected real data from an Oxide class trial. The synthetic dataset from the STNG CT-GAN generator had the highest STNG ML score. The real AUC was 0.92. STNG CT-GAN had a slightly higher STNG ML score than STNG TVAE since STNG TVAE may slightly overfit the synthetic dataset. On the other hand, generic TVAE had an AUCss of 0.89, but its AUCsr was only 0.775, much lower than the real AUC. Therefore, the synthetic dataset from STNG CT-GAN was considered the best synthetic oxide dataset. The univariate cumsum plot suggested consistency between the real and CT-GAN synthetic datasets except for the variable of T and Resist.

In yet another example, synthetic data was generated form empirically collected real data form a NHANES diabetes trial. The output had three classes: normal condition, pre-diabetes and diabetes, whose sizes were 1969, 140 and 328, respectively in the real dataset. Due to relatively small sample size of the pre-diabetes class, the synthetic datasets from the generic TVAE approach generated less than 80 pre-diabetes class (in their synthetic generation arm), which didn't meet the requirement for our Auto-ML evaluation. STNG CT-GAN copula GAN and TVAE had their synthetic AUCs very close to 1. That is, the ML model trained from each synthetic training set had almost perfect separation of the output classes when being applied to the corresponding synthetic test set. However, their synthetic-real AUCs noticeably decreased to about 0.83, which suggested possible overfit in the synthetic ML models. Therefore, the synthetic dataset from the STNG Gaussian copula generator was deemed the best synthetic dataset. These results further demonstrate the advantages of using all three AUCs (i.e., AUCrr, AUCss and AUCsr) to evaluate the ML performance of the synthetic dataset.

### CONCLUSION

Considering the above, the “no-assumption” approach of the present disclosure is able to help address many of the limitations and shortcomings of current traditional approaches. For example, STNG employs a multi-function approach to the generic synthetic generation methods. This multi-function approach is more likely to yield better synthetic datasets than the generic approach. Secondly, the present disclosure provides a score to evaluate synthetic datasets. The ML scores combine the difference in the ML systems trained from the real and synthetic data and the generalization difference from the same model being applied to the real and synthetic validation sets. The derivation of the STNG ML score thus mimics the ML process realistically. Thirdly, the present disclosure embeds an Auto-ML validation module, which trains various classification models automatically. This enables fast and accurate selection and ranking of the synthetic generators. Still further, STNG makes no preliminary assumption about the best synthetic data generators and generates a plurality of synthetic datasets for each real input dataset, which translates to a higher likelihood of acquiring the best performing synthetic data for a given task.

The use of such an approach can drastically facilitate data access, which can expedite current approaches to many studies and help shape the future of healthcare. Additionally, such a platform can help facilitate increased studies in rare diseases and like limited data studies by providing expanded synthetic data or those from collective sites that can be shared much easier within the investigator space. The use of synthetic data according to the method and system described can thus help to enhance our patient care approaches and needs.

While various features are presented above, it should be understood that the features may be used singly or in any combination thereof. Further, it should be understood that variations and modifications may occur to hose skilled in the art to which the claimed examples pertain. Particularly, while the present disclosure is directed toward health care scenarios and data, these methods and systems could be applied to other fields and types of data.

What is claimed is:

1. A method comprising:
obtaining an empirically collected dataset;
generating a synthetic dataset based on the empirically collected dataset;
performing an automated machine learning (Auto-ML) analysis of the synthetic dataset; and
generating an Auto-ML score of the synthetic dataset based on a result of the Auto-ML analysis.

2. The method of claim 1, comprising generating a plurality of synthetic datasets according to a plurality of different synthetic data generation methods,
wherein the Auto-ML analysis is performed, and the Auto-ML score is generated, for each of the plurality of synthetic datasets.

3. The method of claim 2, wherein the plurality of different synthetic data generation methods comprise single function and multi-function models.

4. The method of claim 2, wherein the plurality of synthetic data generation methods comprise single function and multi-function versions of each of Gaussian copula, copula-GAN, CT-GAN, and TVAE models.

5. The method of claim 2, further comprising:
identifying an optimal one of the plurality of synthetic datasets based on the Auto-ML scores.

6. The method of claim 2, further comprising:
validating the plurality of synthetic datasets based on the Auto-ML scores.

7. The method of claim 1, wherein performing the Auto-ML analysis of the synthetic dataset comprises:
training a synthetic data machine learning system with a training portion of the synthetic dataset;
training a real data machine learning system with a training portion of the empirically collected dataset;
inputting a testing portion of the synthetic dataset to the trained synthetic data machine learning system;
inputting a testing portion of the empirically collected dataset to the trained real data machine learning system; and
inputting the testing portion of the empirically collected dataset to the trained synthetic data machine learning system,
wherein the Auto-ML score is based on a comparison of the outputs of the trained real data and synthetic data machine learning systems.

8. The method of claim 7, wherein the Auto-ML score is determined as 1−(|AUCsr−AUCrr|+|AUCss−AUCsr|), where:
AUCsr is a value representing an area under a receiver operating characteristic curve (AUC) of an output of the trained synthetic data machine learning system from inputting the testing portion of the empirically collected dataset;
AUCrr is a value representing an AUC of an output of the trained real data machine learning system from inputting the testing portion of the empirically collected dataset; and
AUCss is a value representing an AUC of an output of the trained synthetic data machine learning system from inputting the testing portion of the synthetic dataset.

9. The method of claim 1, further comprising:
generating a pre-ML score based on a statistical comparison of the empirically collected dataset and the synthetic dataset.

10. The method of claim 1, further comprising:
generating a final score by averaging the Auto-ML score and the pre-ML score.

11. The method of claim 1, wherein generating the synthetic dataset comprises:
splitting the empirically collected dataset into a plurality of classification groups; and
applying a synthetic data generation method to each of the plurality of classification groups.
